(19) United States
(12) Patent Application Publication
(10) Pub. No.: US 2023/0048369 A1
(43) Pub. Date: Feb. 16, 2023

(54) POLYOXAZOLINE MODIFICATIONS MITIGATE STRUCTURAL DEGENERATION OF BIOPROSTHETIC HEART VALVES

(71) Applicants: The Trustees of Columbia University in The City of New York, New York, NY (US); Children's Hospital of Philadelphia, Philadelphia, PA (US)

(72) Inventors: Giovanni Ferrari, Hastings on Hudson, NY (US); Antonio Frasca, Bronx, NY (US); Yingfei Xue, Mountain Lakes, NJ (US); Andrey Zakharchenko, Philadelphia, PA (US); Michael Chorny, Philadelphia, PA (US); Robert Levy, Philadelphia, PA (US); Ivan Alferiev, Philadelphia, PA (US)

(21) Appl. No.: 17/792,694

(22) PCT Filed: Jan. 13, 2021
(86) PCT No.: PCT/US21/13297

§ 371 Date: Jul. 13, 2022

Related U.S. Application Data
(60) Provisional application No. 62/960,644, filed on Jan. 13, 2020.

(51) Int. Cl. A61F 2/24; A61L 27/36

(52) U.S. Cl. CPC A61F 2/24; A61L 27/3625; A61L 2430/20

(57) ABSTRACT

Compositions and methods for mitigating SVD mechanisms in BHV, including non-calcific SVD mechanisms, are provided.

### CROSS-REFERENCE TO RELATED APPLICATIONS

The present application claims the benefit of U.S. Provisional Application Ser. No. 62/960,644 filed 13 Jan. 2020; which is incorporated herein by reference in its entirety.

### STATEMENT REGARDING FEDERALLY SPONSORED RESEARCH OR DEVELOPMENT

This invention was made with government support under NIH grant RO1 HL131872. The government has certain rights in the invention.

### MATERIAL INCORPORATED-BY-REFERENCE

Not Applicable.

### BACKGROUND OF THE INVENTION

Heart valve disease is a common disorder, affecting millions worldwide, and at present cannot be treated medically. Surgery or catheter intervention are the only options for severe heart valve disease, and can involve either repair or more commonly the use of a prosthetic heart valve. Bioprosthetic heart valves (BHV) fabricated from glutaraldehyde-crosslinked heterografts, such as bovine pericardium (BP) or porcine aortic valves (PAV), are widely used to treat severe heart valve disease. BHV are preferred to mechanical prostheses because BHV in general have a lower risk of thrombo-embolic events, and BHV patients typically do not require anticoagulants. BHV at this time are either surgically implanted or deployed with transcatheter techniques.

Despite outstanding short-term outcomes, BHV dysfunction due to structural valve leaflet degeneration (SVD) develops over time, frequently necessitating device replacement. SVD occurs regardless of the heterograft material, requiring prosthesis replacement. Although calcification is observed in the majority of SVD cases, 25% or more SVD cases are not associated with calcification.

Compositions and methods for mitigating SVD mechanisms in BHV, including non-calcific SVD mechanisms, would thus represent an important advancement in the field.

### SUMMARY OF THE INVENTION

The present disclosure provides a method of mitigating structural valve degeneration in a bioprosthetic heart valve, bio-implantable tissue, or another protein based biomaterial for use in surgical or medical therapy, the method comprising treating the bioprosthetic heart valve, bio-implantable tissue or other protein based biomaterial with polyoxazoline in an amount sufficient to prevent or reduce physiological glycation of the bioprosthetic heart valve, bio-implantable tissue, or another protein based biomaterial for use in surgical or medical therapy. In certain embodiments the protein based biomaterial for use in surgical or medical therapy is a cardiac, hernia or pericardial patch, a cardiac conduit, a vascular conduit, a reconstructive surgical implant, an abdominal wall reconstruction, or a material construct used in medicine and surgery comprising a protein as part of its composition.

The present disclosure further provides a method of reducing or preventing structural degeneration in a protein-based biomaterial used in medicine or surgery, comprising covalently attaching a chemical entity to prevent the development of protein infiltration and formation of advanced glycation end products in the protein-based biomaterial used in medicine or surgery. In certain embodiments the protein-based biomaterial used in medicine or surgery is a bioprosthetic heart valve leaflet composed of heterograft or homograft (e.g., cryopreserved human homograft) tissue. In some embodiments the bioprosthetic heart valve leaflet composed of heterograft or homograft tissue is chemically fixed. In other embodiments the chemical entity is an oxidation resistant, biocompatible polymer. In certain embodiments the oxidation resistant, biocompatible polymer is polyoxazoline. In certain embodiments the polyoxazoline is labeled with a fluorescent marker. In particular embodiments the polyoxazoline has a molecular weight of 500 to 500,000 Daltons. In yet other embodiments the polyoxazoline is poly(2-oxazoline), poly-(2-methyl oxazoline), poly(2-ethyl-2-oxazoline), or any poly(2-alkyl-2-oxazoline or poly(2-aryl-2-oxazoline). In further embodiments a combination of two or more different POZ polymers can be used, including, but not limited to, a combination of poly(2-methyl-2-oxazoline and poly(2-ethyl-2-oxazoline), and the combination can use various ratios of the different POZ polymers. In still further embodiments the poly-(2-methyl-2-oxazoline) or poly(2-ethyl-2-oxazoline) has a molecular weight of 500 to 500,000 Daltons.

In certain embodiments, the polyoxazoline has a molecular weight of 500 Daltons, 600 Daltons, 700 Daltons, 800 Daltons, 900 Daltons, 1,000 Daltons, 2,000 Daltons, 3,000 Daltons, 4,000 Daltons, 5,000 Daltons, 6,000 Daltons, 7,000 Daltons, 8,000 Daltons, 9,000 Daltons, 10,000 Daltons, 11,000 Daltons, 12,000 Daltons, 13,000 Daltons, 14,000 Daltons, 15,000 Daltons, 16,000 Daltons, 17,000 Daltons, 18,000 Daltons, 19,000 Daltons, 20,000 Daltons, 25,000 Daltons, 30,000 Daltons, 35,000 Daltons, 40,000 Daltons, 45,000 Daltons, 50,000 Daltons, 60,000 Daltons, 70,000 Daltons, 80,000 Daltons, 90,000 Daltons, 100,000 Daltons, 150,000 Daltons, 200,000 Daltons, 250,000 Daltons, 300,000 Daltons, 350,000 Daltons, 400,000 Daltons, 450,000 Daltons, or 500,000 Daltons, or any combinations of two or more of the foregoing, including, but not limited to, 500 Da POZ plus 5,000 Dalton POZ, 500 Da POZ plus 10,000 Dalton POZ, 2,000 Da POZ plus 5,000 Dalton POZ, 2,000 Da POZ plus 10,000 Dalton POZ, 500 Da POZ plus 25,000 Dalton POZ, or 5,000 Da POZ plus 10,000 Dalton POZ. The combination of different molecular weight POZ can be any ratio of the component POZ polymers effective to reduce or prevent structural degeneration in a protein-based biomaterial used in medicine or surgery, including, but not limited to, a bioprosthetic heart valve.

In particular embodiments the range of the molecular weight of the polyoxazoline is from 500 to 500,000 Daltons, 2,000 to 200,000 Daltons or 5,000 to 50,000 Daltons.

In some embodiments the POZ is activated to provide a chemically active end group for covalent modification. In certain embodiments the chemically active end group of POZ is suitable for direct coupling, including, but not limited to, amino- or carboxythiol coupling (mercapto-, pyridyldithio- or maleimido-terminated) or click chemistry coupling (azide- or alkyne-terminated). In some embodiments POZ has one or two chemically active end group, and in the latter case these groups can be either the same or different (i.e., either homo- or hetero-bifunctional). In particular embodiments the chemically active end group is a primary amine group. In certain embodiments the primary amine is either open at one or both termini (i.e., monoamino- or a diamino-terminated POZ). In further embodiments the POZ is labeled with a fluorescent label, which in certain aspects permits more accurate quantitation of the POZ.

The present disclosure additionally provides a method of reducing or preventing structural valve leaflet degeneration in bioprosthetic heart valve leaflet tissue, comprising covalently attaching polyoxazoline to the bioprosthetic heart valve leaflet tissue. In certain embodiments the polyoxazoline is in an amount sufficient to reduce or prevent protein glycation of the bioprosthetic heart valve leaflet tissue or to reduce or prevent accumulation of a serum protein in the bioprosthetic heart valve leaflet tissue. In particular embodiments the polyoxazoline is labeled with a fluorescent marker. In some embodiments the serum protein is serum albumin. In other embodiments the structural valve leaflet degeneration is caused by advanced glycation end-products mechanisms involving serum protein access to the bioprosthetic heart valve leaflet tissue. In yet other embodiments the reduction in structural valve leaflet degeneration occurs through limiting serum protein access to the bioprosthetic heart valve leaflet tissue. In further embodiments the bioprosthetic heart valve leaflet tissue is located in a human. In particular embodiments the bioprosthetic heart valve leaflet tissue is modified with polyoxazoline prior to implantation in a human.

The present disclosure also provides a method of inhibiting structural valve leaflet degeneration in bioprosthetic heart valve leaflet tissue comprising covalently attaching polyoxazoline to the bioprosthetic heart valve leaflet tissue, and treating the bioprosthetic heart valve leaflet tissue with an anti-calcification composition. In some embodiments the polyoxazoline is labeled with a fluorescent marker. In certain embodiments the bioprosthetic heart valve leaflet tissue is covalently modified with polyoxazoline prior to treatment with the anti-calcification composition. In other embodiments the bioprosthetic heart valve leaflet tissue is covalently modified with polyoxazoline simultaneously with treatment with the anti-calcification composition. In some embodiments the polyoxazoline is in an amount sufficient to reduce or prevent physiological glycation of the bioprosthetic heart valve leaflet tissue or to reduce or prevent accumulation of a serum protein in the bioprosthetic heart valve leaflet tissue. In yet other embodiments the serum protein is serum albumin.

Additionally the present disclosure provides a method of modifying a bioprosthetic heart valve comprising bioprosthetic heart valve leaflet tissue, the method comprising contacting the bioprosthetic heart valve with polyoxazoline, wherein the bioprosthetic heart valve is covalently modified with polyoxazoline. In particular embodiments the polyoxazoline is labeled with a fluorescent marker. In certain embodiments the polyoxazoline is in an amount sufficient to reduce or prevent physiological glycation of the bioprosthetic heart valve leaflet tissue or to reduce or prevent accumulation of a serum protein in the bioprosthetic heart valve leaflet tissue. In some embodiments the serum protein is serum albumin.

The present disclosure further provides a composition comprising a protein-based biomaterial used in medicine or surgery covalently modified with a chemical entity to prevent the development of protein infiltration and formation of advanced glycation end products in the protein-based biomaterial used in medicine or surgery. In certain embodiments the protein-based biomaterial used in medicine or surgery is a bioprosthetic heart valve leaflet composed of heterograft tissue. In some embodiments the chemical entity is an oxidation resistant, biocompatible polymer. In other embodiments the oxidation resistant, biocompatible polymer is polyoxazoline. In certain embodiments the polyoxazoline is labeled with a fluorescent marker. In yet other embodiments the polyoxazoline has a molecular weight of 500 to 500,000 Daltons. In additional embodiments the polyoxazoline has a molecular weight of 10,000 Daltons. In further embodiments the bioprosthetic heart valve leaflet tissue is covalently modified with polyoxazoline in an amount sufficient to reduce or prevent physiological glycation of the bioprosthetic heart valve leaflet tissue or to reduce or prevent accumulation of a serum protein in the bioprosthetic heart valve leaflet tissue. In yet further embodiments the serum protein is serum albumin.

Other objects and features will be in part apparent and in part pointed out hereinafter.

### DESCRIPTION OF THE DRAWINGS

Those of skill in the art will understand that the drawings, described below, are for illustrative purposes only. The drawings are not intended to limit the scope of the present teachings in any way.

FIG. 1 shows effects of POZ-modified BHV leaflets. Panel A shows adhesion of activated THP-1 cells. Panel B shows the effect of POZ modification on TNFa levels produced by THP-1 cells (24 hr) challenged with CML-modified BSA (BSA-CML).

FIG. 2 shows POZ confers both inhibition of BSA Glyoxal entry to BHV and oxidation resistance: Oxidative conditions (14-day exposure to hydrogen peroxide, 10 mM) on stability of the protective effects of BP modification with PEG/POZ against BSA+glyoxal. Panel A: BP exposed to BSA without glyoxal; Panel B: PEG modified, with 5% BSA+50 mM glyoxal; Panel C: Modified with 5 kDa POZ, conditions as shown; Panel D: BSA with glyoxal (50 mM); Panel E: BP modified with 5 kDa PEG, conditions as shown; Panel F: BP with POZ modification. Immunohistochemical staining using an anti-carboxyl methyl lysine antibody (1:5000 dilution) and Dako DAB HRP immunostaining system. Scale bars are 0.5 mm.

FIG. 3 shows glycation via either glyoxal or glyoxal-human serum albumin results in impaired bioprosthetic heart valve hydrodynamic functionality. Flow simulation studies were carried out using clinical grade, tri-leaflet, bovine pericardial bioprosthetic heart valves. Data are shown as percent changes in parameters depicting hydrodynamic functions. Panel A. Effective orifice area (EOA); Panel B. Mean pressure gradient; and Panel C. Peak ejection velocity. *p<0.001.

FIG. 4 shows the results of pulse duplicator studies. POZ modification of BHV leaflets mitigated glycation via glyoxal-human serum albumin. Flow simulation studies were carried out as shown (FIG. 3) using clinical grade, tri-leaflet, bovine pericardial bioprosthetic heart valves. Data are shown as percent changes in parameters depicting hydrodynamic functions, and demonstrating that POZ modification mitigates loss of function due to co-incubations with Glyoxal-HSA. Panel A. Effective orifice area (EOA); Panel B. Mean pressure gradient; Panel C. Peak ejection velocity.

FIG. 5 shows the results of human serum albumin immunostaining of POZ-modified compared to non-modified bovine pericardial bioprosthetic leaflets. First Panel. Unmodified BHV exposed to phosphate buffered saline. Second Panel. Unmodified BHV exposed to human serum albumin. Third Panel. POZ-modified BHV exposed to phosphate buffered saline. Fourth Panel. POZ-modified BHV exposed to human serum albumin.

FIG. 6 shows anti-AGE immunostaining of 21 day rat subdermal BP implants. Results demonstrating mitigation of AGE deposition with POZ. Immuno-peroxidase, original magnification 40×. Panel A. Glutaraldehyde modified control explant. Panel B. POZ-modified explant. Panel C. Explant modified with the EDC conjugation reactions used to covalently attach POZ to BP.

FIG. 7 shows the amount of POZ covalently bound to BP quantified as a function of pre-incubation temperature and expressed as pg POZ/mg BP.

FIG. 8 shows the effect of polyoxazoline (POZ) on the mass uptake by glutaraldehyde-crosslinked bovine pericardium (BP). BSA mass percent change (Uptake, %) for unmodified bovine pericardium (BP) or POZ-modified during 28-day incubation in 5% BSA solution.

FIG. 9 shows the effect of polyoxazoline (POZ) on the incorporation of bovine serum albumin (BSA) by glutaraldehyde-crosslinked bovine pericardium (BP). Immunostaining for BSA of BP tissues either unmodified (Panel i) or POZ modified (Panel ii) incubated in 5% BSA for 3 days. Immunostaining of unmodified control BP prior to BSA incubation (Panel iii).

FIG. 10 shows the effect of oxidative conditions on BP modified with either with polyethylene glycol (PEG) or polyoxazoline (POZ) on TNFα production by THP-1 cells. Bovine pericardium (BP) samples were modified with PEG/POZ and exposed to 10 mM hydrogen peroxide (H₂O₂) for 14 days at physiological conditions followed by 7 days incubation at 50 mM glyoxal+5% BSA.

### DETAILED DESCRIPTION OF THE INVENTION

Applicants have demonstrated that BHV are susceptible to non-calcification induced failure mechanisms, which include inflammatory cell derived, oxidative modifications. Applicants recently observed that SVD is strongly associated with the presence of advanced glycation end-products (AGE) in BHV leaflets. AGE are post-translational, non-enzymatic carbohydrate modifications of proteins. AGE can include both crosslinks, such as glucosepane, and pro-inflammatory ligands that interact with a receptor for AGE (known as RAGE), which is present in all inflammatory cells. Furthermore, glycation profoundly affects serum proteins, and applicants have shown the presence of AGE-modified serum albumin in failed BHV. Applicants have shown that serum albumin infiltration is a major contributor towards BHV glycation, leading to impairment of BHV biomechanics and collagen microarchitecture, as well as a change in BHV thickness and dry mass. Incorporation of glycated proteins on implantable biomaterial leads to enhanced inflammatory cell activation through the receptor for advanced glycation end products (RAGE) and tissue stiffening due to crosslinking. These structural changes ultimately impair BHV hydrodynamic properties. In addition, glycation of lipids is a well-established mechanism involving the same reaction pathways as protein glycation.

Thus the present disclosure is based, at least in part, on the discovery that since BHV leaflets lack living cells or AGE degrading enzymes, they are highly susceptible to accumulation of AGE, glycated lipids, and serum proteins. The present disclosure, described in detail below, uses polyoxazoline (also referred to as poly(2-oxazoline) or poly(2-alkyl/aryl-2-oxazoline); POZ) to reduce glycation of both proteins and lipids, and restrict serum protein uptake. Taken together reducing AGE formation and serum proteins accumulation in BHV results in improvements in the safe and effective use of these devices in long term applications.

As used herein, the term “POZ” will be understood to refer to any of the polyoxazoline class of polymers, including, but not limited to, poly(2-oxazoline), poly(2-methyl-2-oxazoline, poly(2-ethyl-2-oxazoline, or in fact any poly(2-alkyl-2-oxazoline) (linear or branched) or poly(2-aryl-2-oxazoline).

Based on these findings, the present disclosure provides compositions and methods for mitigating the structural degeneration of BHV or any other medical device composed of protein-based materials. The present disclosure also provides a method of modifying bio-implantable tissue or protein based biomaterials, other than BHV. Examples include, but are not limited to, cardiac and pericardial patches, cardiac conduits, vascular conduits, reconstructive surgical implants, abdominal wall reconstruction, and all other material constructs used in medicine and surgery containing proteins as part of their composition.

Additional Age/Rage Therapeutics

The POZ anti-glycation agents described herein can also be used with one or more AGE/RAGE therapeutic agents, including, but not limited to, anti-oxidants, pyridoxamine (Pereira-Simon et al., PLoS One 11:e0159666, 2016; Brodeur et al., PLoS One 9:e85922, 2014), AGE breakers that disrupt AGE structure (Brodeur et al., PLoS One 9:e85922, 2014, Candido et al., Circ. Res. 92:785-792, 2003) and RAGE-specific receptor antagonists (Cai et al., Cell. Mol. Neurobiol. 36:483-495, 2016; Deane et al., J. Clin. Invest. 122:1377-1392, 2012). Additional agents for use with the presently disclosed anti-glycation agents include, but are not limited to, PHOTOFIX® (CryoLife, Inc. Kennesaw, Ga.), pentagalloyl glucose, XLF-III-43 (U.S. Pat. No. 8,729,280), irbesartan, TM2002 (Izuhara et al., Nephrol. Dial. Transplant. 23:497-509, 2007), diclofenac, pioglitazone, metform in, pentoxifylline and N-phenacylthiazolium bromide, as well as compounds disclosed in U.S. Patent Application Publication Number 2014/0127804, International Patent Application Publication Number WO 2013/032969, and U.S. Pat. Nos. 6,093,530, 6,552,077, 10,016,450.

Formulation

The POZ agents and compositions described herein can be formulated by any conventional manner using one or more pharmaceutically acceptable carriers or excipients as described in, for example, Remington's Pharmaceutical Sciences (A.R. Gennaro, Ed.), 21st edition, ISBN: 0781746736 (2005), incorporated herein by reference in its entirety. Such formulations will contain an effective amount of POZ or comparable agent described herein, which can be in purified form, together with a suitable amount of carrier so as to provide the form for proper modification of a BHV or other protein-based biomaterial implant.

Therapeutic Methods for Modifying Bioprosthetic Heart Valves and Other Medical Devices Containing Protein-Based Materials Susceptible to Advanced Glycation Endproduct Formation and Serum Protein Deposition

Also provided is a process of mitigating or treating structural valve degeneration in a subject in need thereof comprising implanting a POZ-modified BHV leaflet, as described herein, in a patient in need thereof, or in certain embodiments administration of a therapeutically effective amount of a POZ anti-glycation agent described herein.

Methods described herein are generally performed on materials to be implanted in a subject in need thereof. A subject in need of the therapeutic methods described herein can be a subject having, diagnosed with, suspected of having, or at risk for developing structural valve degeneration. A determination of the need for treatment will typically be assessed by a history and physical exam consistent with the disease or condition at issue. Diagnosis of the various conditions treatable by the methods described herein is within the skill of the art. The subject can be an animal subject, including a mammal, such as horses, cows, dogs, cats, sheep, pigs, mice, rats, monkeys, hamsters, guinea pigs, and chickens, and humans. For example, the subject can be a human subject.

Generally, a safe and effective amount of a POZ anti-glycation agent incorporated into a BHV or other protein based medical device composite described herein is, for example, that amount that would cause the desired therapeutic effect in a subject while minimizing undesired side effects. In various embodiments, an effective amount of an agent described herein can mitigate structural valve degeneration, for example by substantially inhibiting structural valve degeneration, ameliorating structural valve degeneration, slowing the progress of structural valve degeneration, or limiting the development of structural valve degeneration.

When used in the treatments described herein, a therapeutically effective amount of a POZ anti-glycation agent used to modify a medical device containing protein-biomaterials described herein can be employed in pure form or, where such forms exist, in pharmaceutically acceptable salt form and with or without a pharmaceutically acceptable excipient. For example, the compounds of the present disclosure can be administered, at a reasonable benefit/risk ratio applicable to any medical treatment, in a sufficient amount to mitigate, treat or prevent structural valve degeneration.

Toxicity and therapeutic efficacy of compositions described herein can be determined by standard pharmaceutical procedures in cell cultures or experimental animals for determining the LD₅₀ (the dose lethal to 50% of the population) and the ED₅₀, (the dose therapeutically effective in 50% of the population). The dose ratio between toxic and therapeutic effects is the therapeutic index that can be expressed as the ratio LD₅₀/ED₅₀, where larger therapeutic indices are generally understood in the art to be optimal.

Kits

Also provided are kits. Such kits can include all materials necessary to modify a BHV or other protein-based biomaterial with POZ in a standard and uniform manner. Such kits can include an agent or composition described herein and, in certain embodiments, instructions for administration or testing. Such kits can facilitate performance of the methods described herein. When supplied as a kit, the different components of the composition can be packaged in separate containers and admixed immediately before use. Components include, but are not limited to the anti-glycation agents described herein. Such packaging of the components separately can, if desired, be presented in a pack or dispenser device which may contain one or more unit dosage forms containing the composition. The pack may, for example, comprise metal or plastic foil such as a blister pack. Such packaging of the components separately can also, in certain instances, permit long-term storage without losing activity of the components.

Kits may also include reagents in separate containers such as, for example, sterile water or saline to be added to a lyophilized active component packaged separately. For example, sealed glass ampules may contain a lyophilized component and in a separate ampule, sterile water, sterile saline or sterile each of which has been packaged under a neutral non-reacting gas, such as nitrogen. Ampules may consist of any suitable material, such as glass, organic polymers, such as polycarbonate, polystyrene, ceramic, metal or any other material typically employed to hold reagents. Other examples of suitable containers include bottles that may be fabricated from similar substances as ampules, and envelopes that may consist of foil-lined interiors, such as aluminum or an alloy. Other containers include test tubes, vials, flasks, bottles, syringes, and the like. Containers may have a sterile access port, such as a bottle having a stopper that can be pierced by a hypodermic injection needle. Other containers may have two compartments that are separated by a readily removable membrane that upon removal permits the components to mix. Removable membranes may be glass, plastic, rubber, and the like.

In certain embodiments, kits can be supplied with instructional materials. Instructions may be printed on paper or other substrate, and/or may be supplied as an electronic-readable medium, such as a floppy disc, mini-CD-ROM, CD-ROM, DVD-ROM, Zip disc, videotape, audio tape, and the like. Detailed instructions may not be physically associated with the kit; instead, a user may be directed to an Internet web site specified by the manufacturer or distributor of the kit.

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

All publications, patents, patent applications, and other references cited in this application are incorporated herein by reference in their entirety for all purposes to the same extent as if each individual publication, patent, patent application or other reference was specifically and individually indicated to be incorporated by reference in its entirety for all purposes. Citation of a reference herein shall not be construed as an admission that such is prior art to the present disclosure.

Having described the present disclosure in detail, it will be apparent that modifications, variations, and equivalent embodiments are possible without departing the scope of the present disclosure defined in the appended claims. Furthermore, it should be appreciated that all examples in the present disclosure are provided as non-limiting examples.

### EXAMPLES

The following non-limiting examples are provided to further illustrate the present disclosure. It should be appreciated by those of skill in the art that the techniques disclosed in the examples that follow represent approaches the inventors have found function well in the practice of the present disclosure, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the present disclosure.

### Example 1

### POZ-Modified BHV Leaflets Studies

Formulation Studies

Applicants reasoned that infiltration of proteins, such as serum albumin or alkaline phosphatase into BHV can be mitigated by using POZ as a barrier, which would restrict their entry into BHV. POZ is highly biocompatible, resists oxidation, and has not been shown to be immunogenic. The best known, and clinically used comparable polymer, polyethylene glycol (PEG), while biocompatible, is subject to oxidative degradation, and because of its wide spread use in laxatives, has been shown to be highly immunogenic. It is estimated that more than 70% of adult human subjects have antibodies to PEG. POZ is used exclusively in pharmaceutics. The present disclosure is the first report of using POZ as a barrier to prevent protein infiltration into BHV leaflets.

A biocompatible polymer, poly(2-oxazoline), was attached to bioprosthetic tissue to mitigate the uptake of serum proteins. POZ is a relatively recently synthesized polymer that has not been previously studied for use with bioprosthetic heart valves. Functionalization of bioprosthetic tissue with a dense POZ layer serves as a barrier against uptake of serum proteins and formation of AGE. The BHV leaflet tissue may be preferably a porcine aortic valve or bovine pericardium. The bioprosthetic tissue may be fixed with glutaraldehyde, epoxy compounds, or other less commonly used crosslinkers that will retain unmodified carboxylic groups throughout the tissue.

The polymer used for initial derivatization studies was amine terminated POZ, with a molecular weight of 5,000 Da (obtained from Sigma Aldrich, St. Louis, Mo.). POZ 10,000 Da was obtained from Ultroxa (Belgium, HR11.0115/01.02A). The modification of bioprosthetic tissue with the POZ employed direct coupling of the BHV collagen's carboxylic groups (from residues of aspartic and glutamic acids) with amine end groups of the POZ polymer end chains using water-soluble N-(3-Dimethylaminopropyl)-N′ethylcarbodiimide hydrochloride (EDC). To stabilize short-living intermediate formed in the reaction of carboxylic acid with EDC, N-Hydroxysuccinimide (NHS) was added to the reaction mixture. As a result, more stable amine-reactive ester was formed, and polymer was then bound to the tissue via amide bonds.

The reaction shown below was carried out in an aqueous solution (10 ml) containing 100 mg of POZ [Amine-Poly(2-ethyl-2-oxazoline) and N-Hydroxysuccinimide (NHS, 50 mg).

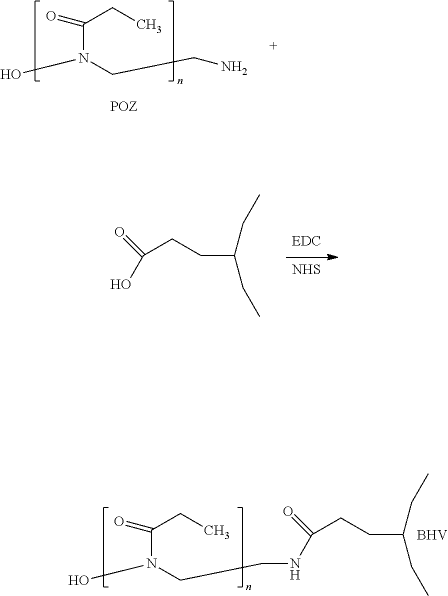

The solution was adjusted to pH 5.5 with 0.05M Potassium bicarbonate, and N-(3-Dimethylaminopropyl)-NLethylcarbodiimide hydrochloride (EDC, 100 mg) was added to initiate the reaction. The BHV samples, glutaraldehyde pretreated bovine pericardium, were allowed to react for 24 hours at room temperature with mild shaking. Finally, the BHV samples were extensively washed to remove unbound polymer and reaction byproducts. After rinsing, the tissue was stored in saline buffer (pH 7.4).

Studies on POZ-Modified BHV Leaflets

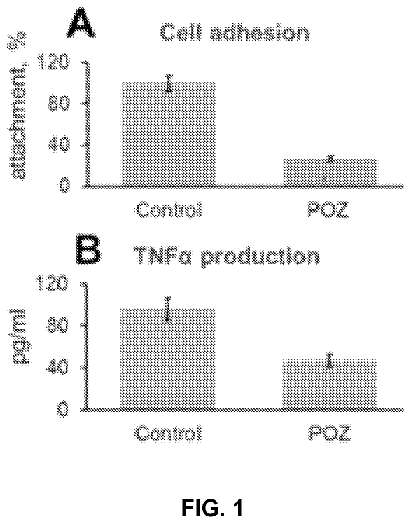

POZ-modified BHV leaflets demonstrated diminished serum albumin uptake, resistance to oxidation, and anti-inflammatory properties. The anti-inflammatory effects of POZ were evaluated using activated human monocytes (THP-1), forming monocyte derived macrophages. Cells were seeded on the surface of control and POZ-modified BP. After 24 hours, samples were washed with PBS and incubated with resazurin (Alamar Blue) for 3 hours to fluorimetrically measure the number of adherent viable cells: a 5-fold decrease in cell adhesion was observed for POZ-modified vs. control BP samples (FIG. 1, Panel A).

Carboxymethyl lysine (CML)-modified albumin has been shown to upregulate the release of inflammatory cytokines from THP-1 cells. BHV leaflets pre-modified with POZ and incubated with CML-modified BSA (BSA-CML) were used as substrates for seeding THP-1 cells. TNFa levels were analyzed in cell culture media samples by ELISA after 24 hr. While POZ-unmodified (control) samples showed a marked increase (215±11%) in TNFα levels, samples modified with POZ did not exhibit quantifiable changes in TNFα production, showing cytokine levels comparable to those seen without the BSA-CML challenge (FIG. 1, Panel B).

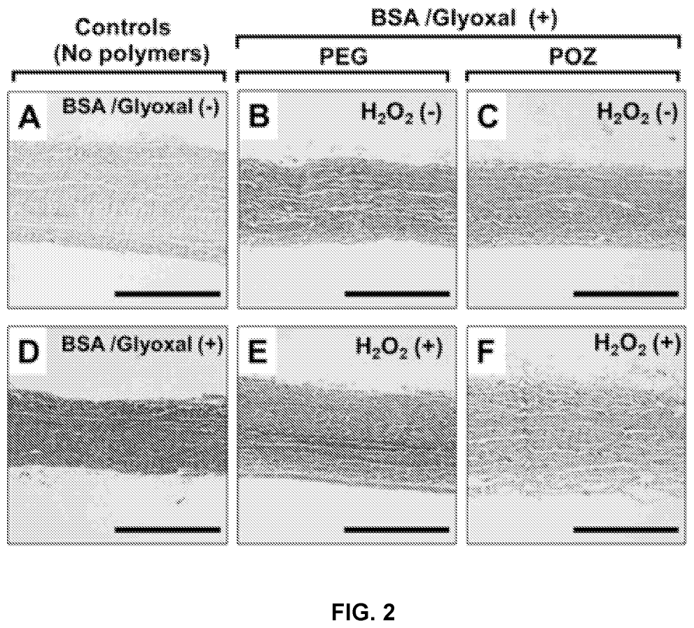

Co-incubation of BHV leaflets with BSA and glyoxal as an initiator of accelerated glycation was evaluated by IHC after staining with an anti-CML antibody. Glutaraldehyde-fixed tissue did not develop any staining (FIG. 2, Panel A), while pre-incubation with BSA/glyoxal showed intense IHC staining for CML (FIG. 2, Panel D). As part of this study, the stability of the protective effect by POZ (5 kDa) under oxidizing conditions was tested in comparison to PEG (5 kDa) using a 14-day exposure to hydrogen peroxide (H₂O₂, 10 mM). H₂O₂-exposed and unexposed samples were simultaneously incubated for another 7 days with BSA/glyoxal at physiological pH, followed by IHC staining for CML. After peroxide exposure, PEG-modified samples exhibited an almost two-fold reduction in the protective effect (FIG. 2, Panel B vs. FIG. 2, Panel E, ImageJ analysis). Remarkably, the protective effect mediated by POZ was completely unaltered by the exposure to peroxide indicating resistance to oxidative stress (FIG. 2, Panel F vs. FIG. 2, Panel C).

Pulse Duplicator Studies

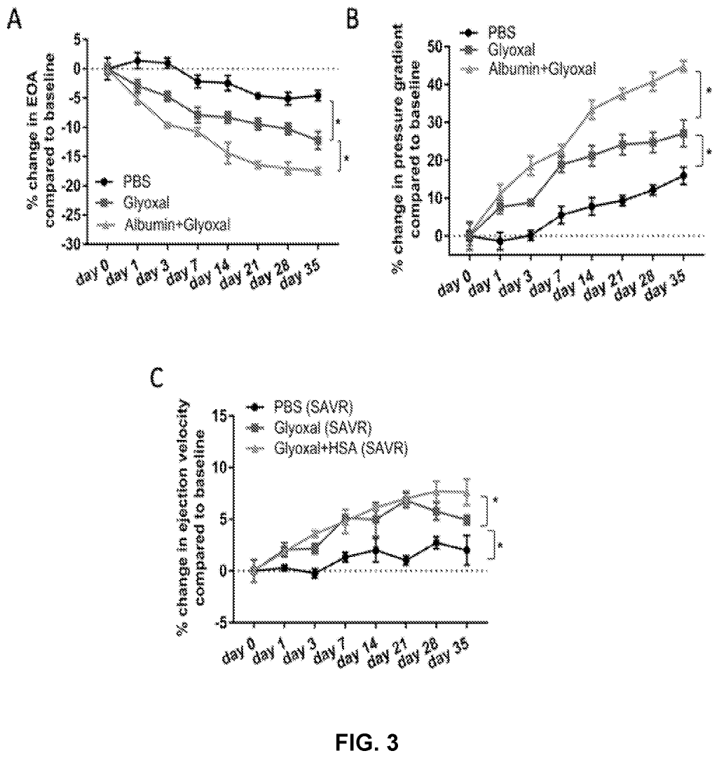

Pulse duplicator studies demonstrated that serum albumin uptake by bioprosthetic heart valve leaflets adversely affects hydrodynamic functionality. Flow simulation studies were carried out using clinical grade, tri-leaflet, bovine pericardial bioprosthetic heart valves, and percent changes in parameters depicting hydrodynamic functions were determined (FIG. 3). These results obtained using an aortic valve pulse duplicator system demonstrate that increased glycation using model incubations for the durations shown of either glyoxal alone or glyoxal plus human serum albumin, resulted in a progressive deterioration of heart valve functionality.

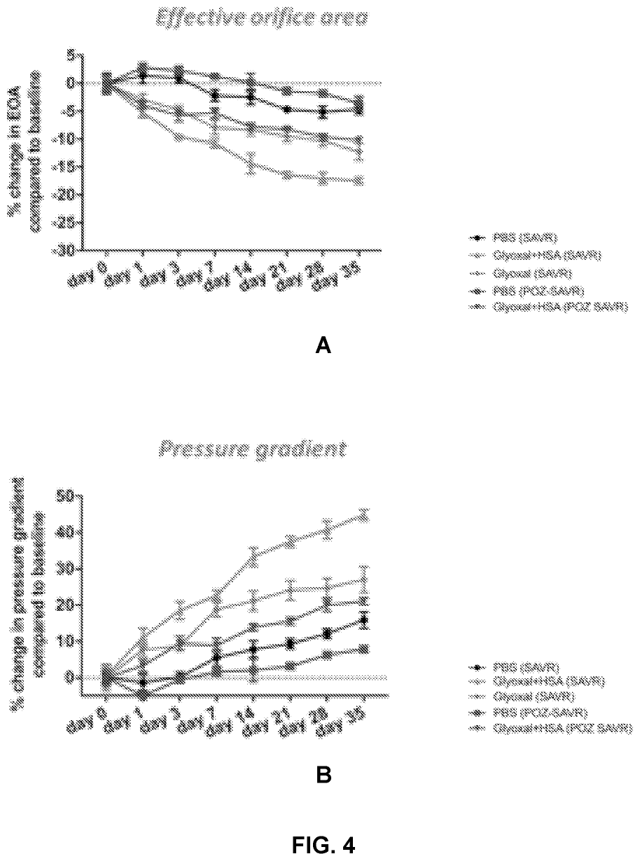

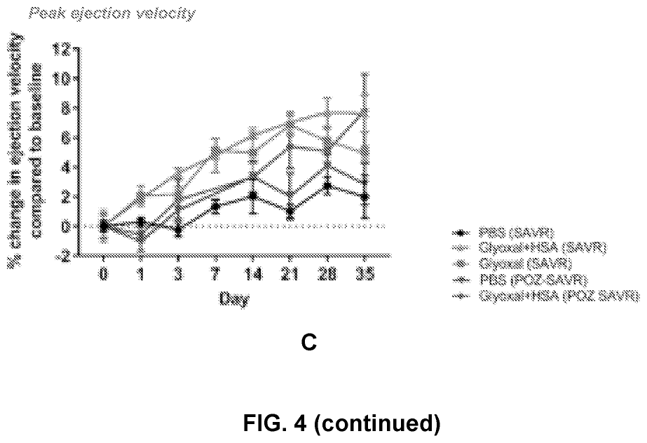

The pulse duplicator studies also demonstrated that POZ modification mitigates the adverse effects of serum albumin uptake by bioprosthetic heart valve leaflets on hydrodynamic functionality. Flow simulation studies were carried out as shown (FIG. 3) using clinical grade, tri-leaflet, bovine pericardial bioprosthetic heart valves. Data are shown as percent changes in parameters depicting hydrodynamic functions, and demonstrated that POZ modification mitigates loss of function due to co-incubations with Glyoxal-HSA (FIG. 4). Immunohistochemistry staining for human serum albumin of bovine pericardial bioprostheses used in the pulse duplicator studies (FIG. 5) demonstrated that POZ modification reduces the level of immunostaining compared to non-POZ modified BHV leaflets by bioprosthetic heart valve leaflets (compare 2nd panel of FIG. 5 to the 4th panel of FIG. 5, showing reduced HSA immunostaining with POZ 10,000 Da modification. BHV leaflets immuno-stained with an anti-HSA antibody were exposed to glyoxal and HSA for a total of 35 days. These results obtained using measurements derived from an aortic valve pulse duplicator system, after 35 day incubations in the solutions indicated, demonstrate that modification of BHV leaflets, as described above with 10,000 Da POZ, mitigated the effects of increased glycation using incubations for the durations shown of either glyoxal alone or glyoxal plus human serum albumin, which in the absence of POZ resulted in a progressive deterioration of heart valve functionality. This protective effect paralleled the restriction of glycated serum albumin uptake as shown above (FIG. 2).

In Vivo Studies

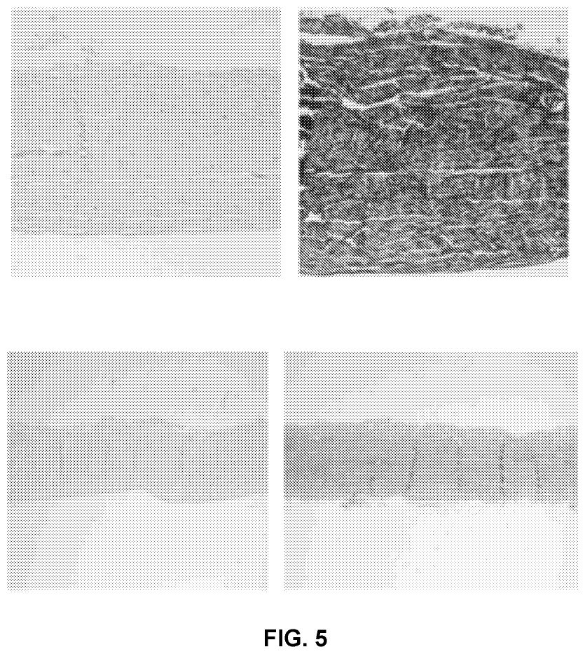

In vivo results with POZ (10,000 Da) modified glutaraldehyde-pretreated bovine pericardium (BP)-rat subdermal implants demonstrated reduced AGE per anti-AGE immunostaining (IHC) results (FIG. 6): As indicated, POZ-modified BP were implanted in juvenile rats as subdermal implants, and retrieved after 28 days. IHC results demonstrated reduced immune-peroxidase staining using an anti-AGE antibody, compared to both a glutaraldehyde modified control explant, and an explant modified with the EDC-conjugation reactions used to covalently attach POZ to BP.

As shown herein, POZ-modification of BHV leaflets with covalent attachment of this polymer has been successfully achieved, and conferred resistance over time to the deleterious uptake of serum proteins by BHV leaflets, that contributes to glycation related SVD. This effect was achieved optimally with 10,000 Da POZ. Inhibition of AGE deposition by POZ has been demonstrated in vitro and in vivo. In addition, a reduced macrophage inflammatory response was demonstrated in vitro. Pulse duplicator studies demonstrated both mitigation of the deleterious effects of glyoxal and HSA on BHV hydrodynamics, and diminished HSA deposition.

### Example 2

### POZ Fluorescent Labeling Studies

POZ Labeling with Fluorescent Dye for Quantitative Binding Analysis In order to determine the amount of POZ bound to bioprosthetic tissues, POZ was functionalized with a fluorescent probe, 5(6)-carboxyfluorescein, as shown in the schematic below.

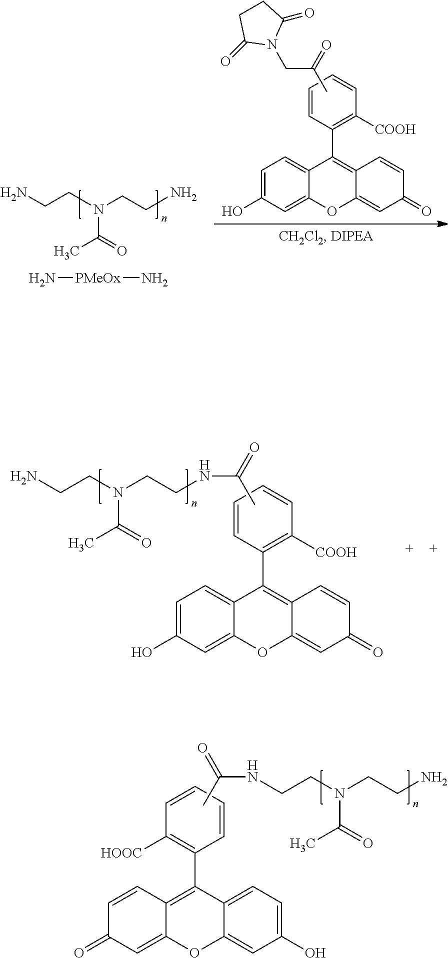

Amine-initiated/amine-terminated poly-(2-methyl-2-oxazoline) (H₂N-PMeOX-NH₂, Ultroxa, Belgium, Mₙ=8700 Da, 200 mg, 0.045 mmol of NH₂) was dissolved in dichloromethane (4 mL) and concentrated to 2.34 g. N-Ethyldiisopropylamine (DIPEA, Sigma-Aldrich, 99.5%, 7 mg, 0.05 mmol) was introduced, and 5(6)-carboxyfluorescein N-hydroxysuccinimide ester (Sigma, ≥80%, 4 mg, ≥0.007 mmol) was added under a vigorous stirring. The mixture was stirred at room temperature for 26 h, becoming homogeneous in 10 min. The solvent was removed (RE, 30° C.), the residue was evacuated (oil pump, room temperature) for 0.5 h, dissolved in water (20 mL), made alkaline with 19 M aqueous NaOH (0.1 mL), and Na₂SO₄ (5.70 g) was added at 30-35° C. After a complete dissolution of the salt, benzene (24 mL) and dichloromethane (6 mL) were added, the mixture was vigorously stirred for 0.5 h, until the polymer was completely coagulated and collected as flocks between the aqueous and the organic phases. The aqueous phase (containing the unbound fluorophore) was removed, the organic phase together with the flocks of polymer was washed in two portions with a solution of Na₂SO₄ (6.00 g) in water (22 mL), dried (RE, 30° C.), and the polymer was extracted with methanol (35 mL). After filtering from the crystals of Na₂SO₄ and drying, the polymer was treated as above with the alkaline aqueous Na₂SO₄ two more times, removing the unbound fluorophore as much as possible. Finally, the polymer was dissolved in dichloromethane (10 mL), filtered, concentrated to 1.93 g and precipitated with methyl-t-butyl ether (MTBE, 3 mL). After filtration, washing with MTBE (20 mL), with hexane (15 mL) and drying in vacuo, the yield of polymer was 181 mg.

Fluorimetric Quantification of POZ Binding to BP Tissue (FIG. 2):

The attachment of POZ to BP was quantified by fluorimetry using a 5/6-carboxyfluorescein labeled fluorescent POZ derivative (F-POZ), as shown in the schematic below.

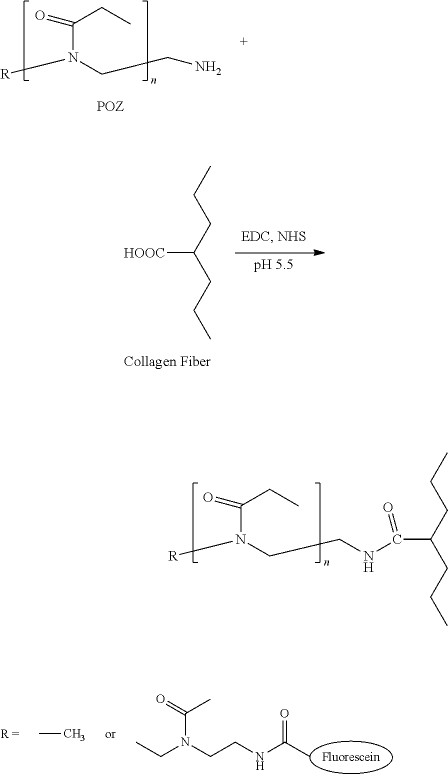

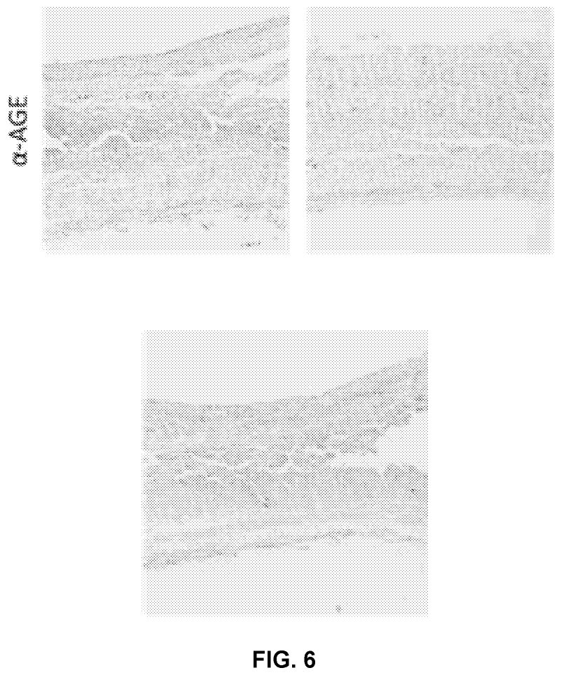

Fresh BP was treated with 0.625% glutaraldehyde in HEPES buffer (50 mM HEPES, 0.9% NaCl, pH 7.4) for 7 days at room temperature, and transferred into 0.2% glutaraldehyde HEPES solution and stored at 4° C. Ten glutaraldehyde-fixed BP tissue samples (1×1 cm) were incubated in a 1 ml (10 mg) mixture of POZ and purified F-POZ (mixed at a 4:1 ratio) overnight at room temperature. Incubations were carried under different temperatures (4° C., 25° C., 37° C.) to find the optimal conditions for the highest tissue saturation with POZ. Then, 43 mM N-hydroxysuccinimide (NHS) and 65 mM of 1-ethyl-3-(3-dimethylaminopropyl) carbodiimide hydrochloride (EDC) were added to each incubation group in 1 ml MES buffer (100 mM 2-(N-morpholino) ethanesulfonic acid, pH 5.5) to start the reaction. The reaction proceeded over 24 hours at room temperature. To quantify attached POZ, modified tissues were triple rinsed with deionized water (5 minutes each), freeze-dried for 24 hours, weighed, and solubilized with Biosol© (National Diagnostics, Atlanta, GA) at 50° C. with shaking for 72 h. The attached polymer was quantified against a suitable calibration curve using λex/λem=490 nm/515 nm and expressed as pg/mg BP (FIG. 7). Quantitative analysis of POZ binding showed that conducting the reaction for 24 hr at 25° C. allows sufficient tissue saturation with polymer, yielding 22+3 μg POZ per gram tissue.

### POZ-Modified BHV Leaflets Demonstrate Diminished Serum Albumin Uptake

The efficiency of BP protection by conjugated POZ against accumulation of serum proteins within the tissue was examined by incubating BP tissue samples (with or without POZ modification) in 5% w/v bovine serum albumin (BSA), a model serum protein that is susceptible to glycation and accumulation in BHV leaflets. Glutaraldehyde-fixed BP samples (1×1 cm) were modified with 10 kDa POZ by placing in 1 ml MES buffer solution with 10 mg of POZ overnight at room temperature.

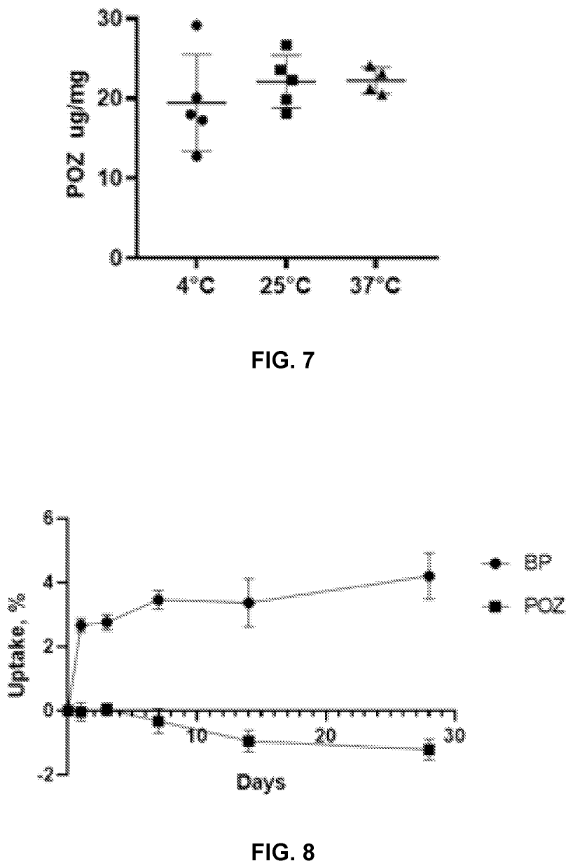

To initiate the reaction, 43 mM N-hydroxysuccinimide (NHS) and 65 mM of 1-ethyl-3-(3-dimethylaminopropyl) carbodiimide hydrochloride (EDC) were added in 1 ml MES resulting in total volume 2 ml, as detailed above. The reaction proceeded for 24 h at room temperature. Samples were triple rinsed with MES buffer, followed by a rinse with deionized water (5 minutes), lyophilized for 24 hours, and their dry weight was recorded after 30 min equilibration at room temperature. Albumin uptake was quantified as the change in the dry weight of the samples over 28 days. Incubation of control BP samples in BSA resulted in a gradual increase in their weight, which could be detected at 24 hours and plateaued at 3.5% after 7 days (FIG. 8). By contrast, POZ functionalization inhibited albumin uptake, with no change in the tissue dry weight during the first 7 days and a small loss of weight between days 7 and 28.

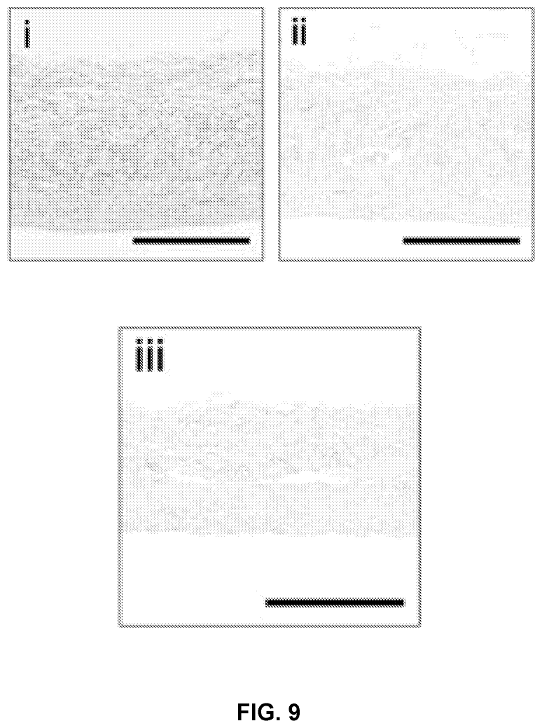

Immunostaining for BSA following 3 days of exposure demonstrated that unmodified BP accumulated albumin uniformly throughout the tissue (FIG. 9, Panel i). In contrast, POZ-modified samples did not accumulate albumin (FIG. 9, Panel ii) and stained similar to unexposed control (FIG. 9, Panel; iii), demonstrating that POZ functionalization of BP collagen tissues can effectively block the uptake of serum proteins.

POZ-Modified BP Tissues Mitigate AGE-Induced Inflammatory Response

The protective effect of BP POZ modification against deleterious effects of glycation was investigated. Modification of tissues with POZ against AGE-mediated inflammatory response was comparatively tested with polyethylene glycol (PEG), a polymer traditionally used to create antifouling surfaces. Samples were cut using 8-mm biopsy punches and functionalized with PEG or POZ using identical conjugation reaction conditions: discs were incubated in 1 ml polymer solutions for 24 hours followed by addition of 43 mM NHS and 65 mM of EDC in 1 ml MES buffer (100 mM 2-(N-morpholino) ethanesulfonic acid, pH 5.5). Stability of the protective effect by POZ under oxidizing conditions was tested in comparison to PEG using a 14-day exposure to hydrogen peroxide (10 mM, in PBS solution at 37° C.) followed by accelerated glycation and co-incubation with macrophages. After exposure to hydrogen peroxide, samples were incubated in vitro in solution 5% (w/v) BSA with 50 mM glyoxal (GLX) (Sigma Aldrich, St. Louis, Mo.) for 7 days at 37° C. in the dark. Resulting accelerated glycation led to formation of carboxymethyl-lysine (CML), a ligand for RAGE receptor present on the surface of most inflammatory cells.

Glycated samples, either unmodified or modified with one of the polymers (PEG/POZ), were used as substrates for activated human macrophages (THP-1). Human monocytes, (THP-1 line), were cultured in RPMI medium, supplemented with 10% fetal bovine serum and 1° A Penicillin-Streptomycin. Monocytes were differentiated to monocyte derived macrophages using 100 ng/ml phorbol 12-myristate 13-acetate and seeded on BP surfaces using the same medium composition. Cell culture was maintained in a 37° C. incubator with 5% CO₂ under a humidified atmosphere. Experiments were conducted at a cell concentration of 2×10⁵ cells/mL. THP-1 cells were seeded on BP discs (8 mm in diameter). Discs were placed in 48-well culture plates with a pericardial side facing up and cells were seeded in PMA-containing media resulting in cells activation. After 24 hours of incubation with cells, the medium was collected to measure TNF-α using a commercially available enzyme-linked immunosorbent assay (ELISA) according to manufacturer's instructions by measuring the optical density at 450 nm.

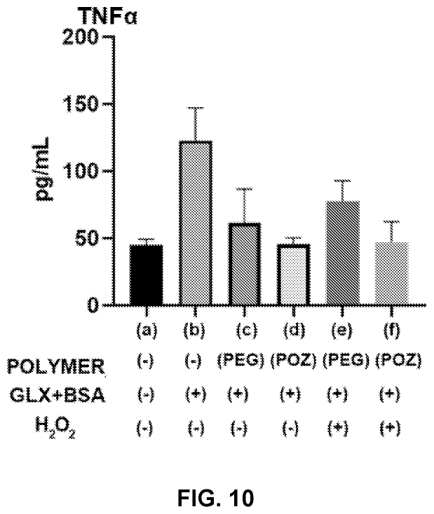

Glycated samples, glutaraldehyde crosslinked bovine pericardium (BP) exposed to bovine serum albumin (50 mg/ml) and GLX (50 mM), abbreviated GLX+BSA, were incubated with THP-1 macrophages showed elevated TNFα levels, compared to unmodified control (FIG. 10, (b) vs (a)). Polymer modification resulted in inhibition of TNFa release in PEG- and POZ-modified samples (FIG. 10, (c), (d)) relative to unmodified control (FIG. 10, (b)). Peroxide incubation prior to glycation exposure resulted in a loss of PEG-mediated anti-inflammatory protection (FIG. 10, (c) vs (e)), whereas the protective effect of POZ was retained (FIG. 10, (d) vs (f)). This data demonstrate that chemically attached POZ provides a unique combination of strong protective effect against inflammatory AGE and chemical stability from oxidative damage.

As shown herein, BHV leaflets modification with covalently attached POZ has been successfully achieved, and confers resistance over time to the deleterious uptake of serum proteins by BHV leaflets, which contributes to glycation related SVD. This effect was achieved optimally with 10,000 molecular weight POZ. Inhibition of AGE deposition by POZ has been demonstrated in vitro and in vivo. In addition, a reduced macrophage inflammatory response was demonstrated in vitro. Protective stability of POZ remained unchanged after 14-days exposure to oxidative conditions.

What is claimed is:

1. A method of reducing or preventing structural degeneration in a protein-based biomaterial used in medicine or surgery, comprising covalently attaching a chemical entity to prevent the development of protein infiltration and formation of advanced glycation end products in the protein-based biomaterial used in medicine or surgery.

2. The method of claim 1, wherein the protein-based biomaterial used in medicine or surgery is a bioprosthetic heart valve leaflet composed of heterograft or homograft tissue.

3. The method of claim 2, wherein the bioprosthetic heart valve leaflet composed of heterograft or homograft tissue is chemically fixed.

4. The method of claim 1, wherein the chemical entity is an oxidation resistant, biocompatible polymer.

5. The method of claim 4, wherein the oxidation resistant, biocompatible polymer is polyoxazoline.

6. The method of claim 5, wherein the polyoxazoline has a molecular weight of 5,000 to 50,000 Daltons.

7. The method of claim 5, wherein the polyoxazoline has a molecular weight of 10,000 Daltons.

8. The method of claim 5, wherein the polyoxazoline is poly-(2-methyl oxazoline) or poly(2-ethyl-2-oxazoline).

9. The method of claim 8, wherein the poly-(2-methyl-2-oxazoline) or poly(2-ethyl-2-oxazoline) has a molecular weight of 5,000 to 50,000 Daltons.

10. The method of claim 5, wherein the polyoxazoline is labeled with a fluorescent marker.

11. A method of reducing or preventing structural valve leaflet degeneration in bioprosthetic heart valve leaflet tissue, comprising covalently attaching polyoxazoline to the bioprosthetic heart valve leaflet tissue.

12. The method of claim 11, wherein the polyoxazoline is in an amount sufficient to reduce or prevent protein glycation of the bioprosthetic heart valve leaflet tissue or to reduce or prevent accumulation of a serum protein in the bioprosthetic heart valve leaflet tissue.

13. The method of claim 12, wherein the serum protein is serum albumin.

14. The method of claim 11, wherein the structural valve leaflet degeneration is caused by advanced glycation end-products mechanisms involving serum protein access to the bioprosthetic heart valve leaflet tissue.

15. The method of claim 11, wherein the reduction in structural valve leaflet degeneration occurs through limiting serum protein access to the bioprosthetic heart valve leaflet tissue.

16. The method of claim 11, wherein the bioprosthetic heart valve leaflet tissue is located in a human.

17. The method of claim 11, wherein the bioprosthetic heart valve leaflet tissue is modified with polyoxazoline prior to implantation in a human.

18. The method of claim 17, wherein the polyoxazoline is labeled with a fluorescent marker prior to implantation in a human.

19. The method of claim 1, wherein the protein-based biomaterial used in medicine or surgery is a bioprosthetic heart valve, a bio-implantable tissue, a cardiac patch, a hernia patch, a pericardial patch, a cardiac conduit, a vascular conduit, a reconstructive surgical implant, an abdominal wall reconstruction, or a material construct used in medicine and surgery comprising a protein as part of its composition.

20. A method of inhibiting structural valve leaflet degeneration in bioprosthetic heart valve leaflet tissue comprising covalently attaching polyoxazoline to the bioprosthetic heart valve leaflet tissue, and treating the bioprosthetic heart valve leaflet tissue with an anti-calcification composition.

21. The method of claim 20, wherein the bioprosthetic heart valve leaflet tissue is covalently modified with polyoxazoline prior to treatment with the anti-calcification composition.

22. The method of claim 20, wherein the bioprosthetic heart valve leaflet tissue is covalently modified with polyoxazoline simultaneously with treatment with the anti-calcification composition.

23. The method of claim 20, wherein the polyoxazoline is in an amount sufficient to reduce or prevent physiological glycation of the bioprosthetic heart valve leaflet tissue or to reduce or prevent accumulation of a serum protein in the bioprosthetic heart valve leaflet tissue.

24. The method of claim 23, wherein the serum protein is serum albumin.

25. The method of claim 20, wherein the polyoxazoline is labeled with a fluorescent marker.

26. A method of modifying a bioprosthetic heart valve comprising bioprosthetic heart valve leaflet tissue, the method comprising contacting the bioprosthetic heart valve with polyoxazoline, wherein the bioprosthetic heart valve is covalently modified with polyoxazoline.

27. The method of claim 26, wherein the polyoxazoline is in an amount sufficient to reduce or prevent physiological glycation of the bioprosthetic heart valve leaflet tissue or to reduce or prevent accumulation of a serum protein in the bioprosthetic heart valve leaflet tissue.

28. The method of claim 26, wherein the serum protein is serum albumin.

29. The method of claim 26, wherein the polyoxazoline is labeled with a fluorescent marker.

30. A composition comprising a protein-based biomaterial used in medicine or surgery covalently modified with a chemical entity to prevent the development of protein infiltration and formation of advanced glycation end products in the protein-based biomaterial used in medicine or surgery.

31. The composition of claim 30, wherein the protein-based biomaterial used in medicine or surgery is a bioprosthetic heart valve leaflet composed of heterograft tissue.

32. The composition of claim 30, wherein the chemical entity is an oxidation resistant, biocompatible polymer.

33. The composition of claim 32, wherein the oxidation resistant, biocompatible polymer is polyoxazoline.

34. The composition of claim 33, wherein the polyoxazoline is labeled with a fluorescent marker.

35. The composition of claim 33, wherein the polyoxazoline has a molecular weight of 500 to 50,000 Daltons.

36. The composition of claim 33, wherein the polyoxazoline has a molecular weight of 10,000 Daltons.

37. The composition of claim 33, wherein the bioprosthetic heart valve leaflet tissue is covalently modified with polyoxazoline in an amount sufficient to reduce or prevent physiological glycation of the bioprosthetic heart valve leaflet tissue or to reduce or prevent accumulation of a serum protein in the bioprosthetic heart valve leaflet tissue.

38. The composition of claim 37, wherein the serum protein is serum albumin.
